(19) United States
(12) Patent Application Publication
(10) Pub. No.: US 2026/0334826 A1
(43) Pub. Date: Oct. 29, 2026

(54) SPO2 EMITTER ARRANGEMENT

(71) Applicant: Polar Electro Oy, Kempele (FI)

(72) Inventors: Joni KILPIJÄRVI, Kempele (FI); Matti KINNUNEN, Kempele (FI); Nuutti SANTANIEMI, Kempele (FI); Seppo KORKALA, Kempele (FI)

(73) Assignee: Polar Electro Oy, Kempele (FI)

(21) Appl. No.: 19/123,414

(22) PCT Filed: Oct. 27, 2023
(86) PCT No.: PCT/FI2023/050602

§ 371 Date: Apr. 23, 2025

(30) Foreign Application Priority Data
Oct. 28, 2022 (GB) 2215977.6

(51) Int. Cl. A61B 5/1455; A61B 5/00

(52) U.S. Cl. CPC A61B 5/14552; A61B 5/681

(57) ABSTRACT

This document discloses an apparatus for optically measuring blood oxygenation from a skin tissue, comprising: a photo detector; a first pair of light emitters comprising a first light emitter configured to emit light at a first wavelength and a second light emitter configured to emit light at a second wavelength different from the first wavelength; a second pair of light emitters comprising a third light emitter configured to emit light at the first wavelength and a fourth light emitter configured to emit light at the second wavelength, wherein the second pair of light emitters is disposed in a substantially perpendicular orientation with respect to the first pair of light emitters to form a T shape or an L shape, and wherein the first pair of emitters and the second pair of emitters are to the same direction i.e. arranged on the same side, from the photo detector.

### CROSS-REFERENCE TO RELATED APPLICATIONS

This application is a national phase application of International Application No. PCT/FI2023/050602, filed Oct. 27, 2023, which claims benefit and priority to Great Britain Application No. 2215977.6, filed Oct. 28, 2022, which are incorporated by reference herein in their entireties.

### BACKGROUND

### Technical Field

The invention relates to a field of wearable training computers and, particularly, to an emitter arrangement for a pulse oximetry sensor.

### SUMMARY

A pulse oximeter also called an SpO2 sensor detects changes in blood oxygen levels non-invasively via optical measurements. Conventionally, light emission on two different wavelengths is needed for measuring the blood oxygenation, and the two wavelengths are conventionally on red and infrared ranges of the spectrum.

The present invention is defined by the subject matter of the independent claim.

Embodiments are defined in the dependent claims.

The embodiments and features, if any, described in this specification that do not fall under the scope of the independent claim are to be interpreted as examples useful for understanding various embodiments of the invention.

### BRIEF DESCRIPTION OF THE DRAWINGS

In the following the invention will be described in greater detail by means of preferred embodiments with reference to the attached drawings, in which

FIGS. 1A and 1B illustrate a wearable sensor device comprising a blood oximetry sensor head according to an embodiment;

FIG. 2 illustrates a T-shaped blood oximetry emitter arrangement according to an embodiment;

FIG. 3 illustrates a modification of the T-shaped blood oximetry emitter arrangement;

FIGS. 4 and 5 illustrate further modifications of the T-shaped blood oximetry emitter arrangement;

FIG. 6 illustrates an angled version of the T-shaped blood oximetry emitter arrangement;

FIG. 7 illustrates an L-shaped blood oximetry emitter arrangement; and

FIG. 8 illustrates a measurement arrangement and procedure for measuring the blood oximetry.

### DETAILED DESCRIPTION

The following embodiments are exemplifying. Although the specification may refer to “an”, “one”, or “some” embodiment(s) in several locations of the text, this does not necessarily mean that each reference is made to the same embodiment(s), or that a particular feature only applies to a single embodiment. Single features of different embodiments may also be combined to provide other embodiments.

Embodiments of the invention relate to a wearable sensor device configured to carry out measurements on a user. In particular, the wearable sensor device is configured to carry out blood oximetry measurements non-invasively via optical measurements. The wearable training computer may be a portable system attachable to the user's body, e.g. to the wrist, arm, torso, or head. The wearable training computer may be configured to measure the blood oximetry of the user during a physical exercise, sleep, and/or during daily activities of the user and to output information on the measured blood oximetry to the user via a user interface of the wearable sensor device and/or via a user interface of another apparatus. In an embodiment, the wearable sensor device has no user interface, and the information is first delivered wirelessly to a training computer for the display. In such a case, the information on the blood oximetry may be output via a wireless interface, e.g. a radio interface complying with a determined radio communication protocol such as a Bluetooth® protocol or another IEEE 802.15-based protocol, or another standardized or proprietary radio communication protocol.

In an embodiment, the wearable training computer may further comprise an apparatus configured to be attached to the object. Such an apparatus may comprise an attachment structure designed and arranged to receive the training computer in a fixed, integrated, or detachable manner and to attach the training computer to the object. The attachment may be realized by a band that may be designed to encircle the object such that the band is attached around the object. The band may comprise locking parts at ends of the band where the locking parts form mutually counterparts such as a buckle and a catch. The locking parts may fix the band around the object as is commonly known in the field of wristwatches, wrist computers etc. Other forms of attachment of wearable devices are equally possible, e.g. the training computer may be integrated or attached to a garment such as a shirt, pants, harness, strap, or headwear.

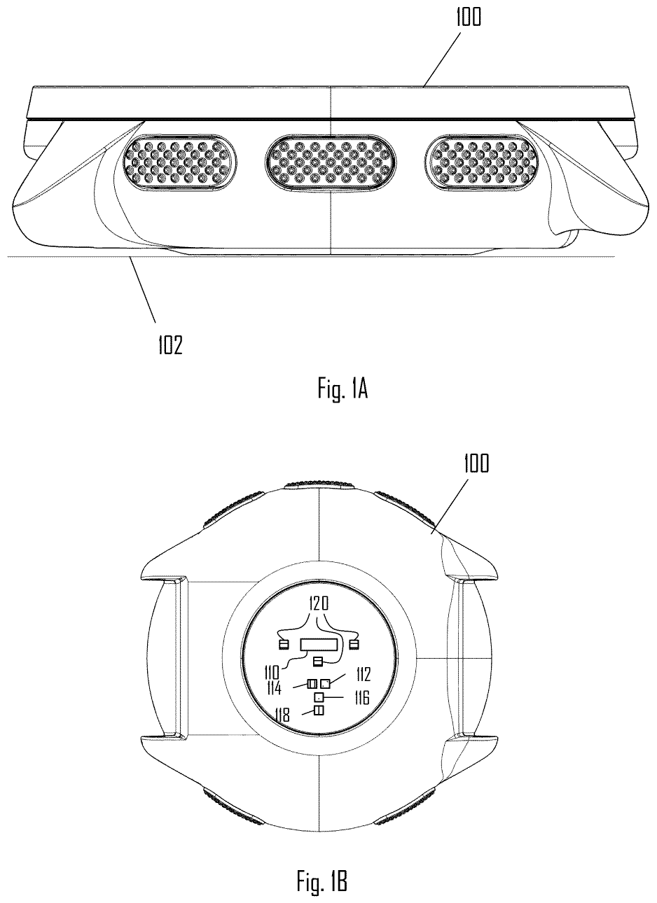

FIGS. 1A and 1B illustrate such a wearable sensor device 100 in the form of a wrist-worn training computer that is illustrated as an example embodiment. FIG. 1A illustrates a side view and FIG. 1B a bottom view of the device 100. On a bottom side of the wearable sensor device 100 that faces the user's skin 102, when the device 100 is worn by the user, there is an optical measurement head for the blood oximetry measurements.

In an embodiment, there is provided an apparatus for the wearable sensor device 100, comprising: a photo detector 110; a first pair of light emitters comprising a first light emitter 112 configured to emit light at a first wavelength (illustrated by dotted pattern) and a second light emitter 116 configured to emit light at a second wavelength (illustrated by vertical lining) different from the first wavelength; a second pair of light emitters comprising a third light emitter 116 configured to emit light at the first wavelength and a fourth light emitter 118 configured to emit light at the second wavelength. The second pair of light emitters is disposed in a substantially perpendicular orientation with respect to the first pair of light emitters to form a T shape or an L shape, and wherein the first pair of emitters and the second pair of emitters are to the same direction from the photo detector 110. The apparatus further comprises a measurement circuitry configured to:
control the first light emitter 112 and the third light emitter 116 to emit light on the first wavelength, and control the photo detector 110 to measure the light on the first wavelength and to generate a first measurement signal on the first wavelength,
control the second light emitter 114 and the fourth light emitter 118 to emit light on the second wavelength, and control the photo detector 110 to measure the light on the second wavelength and to generate a second measurement signal on the second wavelength,
compute a blood oxygenation parameter on the basis of the first measurement signal and the second measurement signal and to output the blood oxygenation parameter through an interface. As described above, the interface may be a user interface or a wireless interface. A yet another embodiment is an internal interface of the wearable sensor device, e.g. an application programming interface.

An advantage provided by the T-shape and arrangement of the light emitters 112 to 118 on the same side of the photo detector is that they illuminate the same tissue and, further, both length and width dimensions can be utilized optimally for the illuminated tissue. Different users have different tissue properties and different dimensions in their limbs (e.g. arms). Therefore, it may be beneficial to realize multiple varying measurement dimensions so that the measurements can be conducted with high performance. For example, it has been observed in connection with blood oxygenation measurements that the infrared light emitter benefits from a greater distance to the photo detector than the red light emitter. The benefit is observed in greater AC-to-DC (alternating current to direct current) ratio in the measurement signal. The blood oxygenation is based on measuring the AC component while the DC component is noise. Therefore, greater AC-to-DC ratio means greater measurement accuracy. Therefore, in several embodiments described below and illustrated in Figures, if the red and infrared light emitters are in a pair of light emitters such that one is behind the other when viewed from the photo detector, it is the infrared light emitter that is behind the red light emitter, thus providing for the greater distance to the photo detector.

In embodiments where the measurement circuitry controls the light emitters of the same wavelength to emit light simultaneously, a further advantage can be gained by the virtue of providing a greater light intensity than a single light emitter per wavelength. The greater light intensity enables reduction of a sampling rate by the measurement circuitry which reduces the power consumption of the apparatus. The T-shape as described above provides an arrangement where all light emitters illuminate substantially the same tissue and yet providing a compact light emitter arrangement.

As illustrated in FIG. 1B and described above, the blood oximetry measurement head comprises the photo detector 110 and the emitter arrangement comprising the light emitters 112 to 118 that are to the same general direction from the photo detector 110. This may be understood such that the light emitters 112 to 118 are on the same side of the photo detector 110 or in the same quadrant from the photo detector. Yet another definition of the arrangement is that the light emitters 112 to 118 face the same side of the photo detector. The photo detector may have a rectangular shape which is advantageous for reducing ambient light noise. The first pair of light emitters 112, 116 and the second pair of light emitters 114, 118 may then all face the same one of the longer sides of the rectangular photo detector 110.

In an embodiment, the apparatus comprises further light emitters 120 for measuring heart activity such as heart rate via photoplethysmography (PPG). The further light emitters may be configured to emit light at a third wavelength (illustrated by horizontal lining) different from the first and second wavelengths. The third wavelength may be in a green light range of the light spectrum. The further light emitters may be disposed on different sides of the photo detector, as illustrated in FIG. 1B. Some of the further light emitters may be on the same side of the photo detector 110 as the light emitters 112 to 118, while some of the further light emitters may be on a different side or different sides of the photo detector 110 than the light emitters 112 to 118.

With respect to the orientation of the blood oximetry measurement head in a wearable training computer configured to be attached to the user's limb, it has been discovered that it is beneficial to arrange the blood oximetry measurement channels to be along the longitudinal dimension of the limb. Referring to FIG. 1B, the direction of the light from the light emitters 112 to 118 to the photo detector along a plane where the light emitters are disposed is parallel to the limb. The casing illustrated in FIG. 1B is oriented so that a strap attaching the casing to the limb extends to the left and to the right from the casing. The light from the light emitters 112 to 118 thus propagates in down-up direction while also travelling through the skin (towards the reader of FIG. 1B).

The light emitters 112 to 120 may be light emitting diodes (LEDs) of laser diodes, for example, and the photo detector may be a photo diode, a charge-coupled device, or a complementary metal-oxide semiconductor (CMOS) detector, for example.

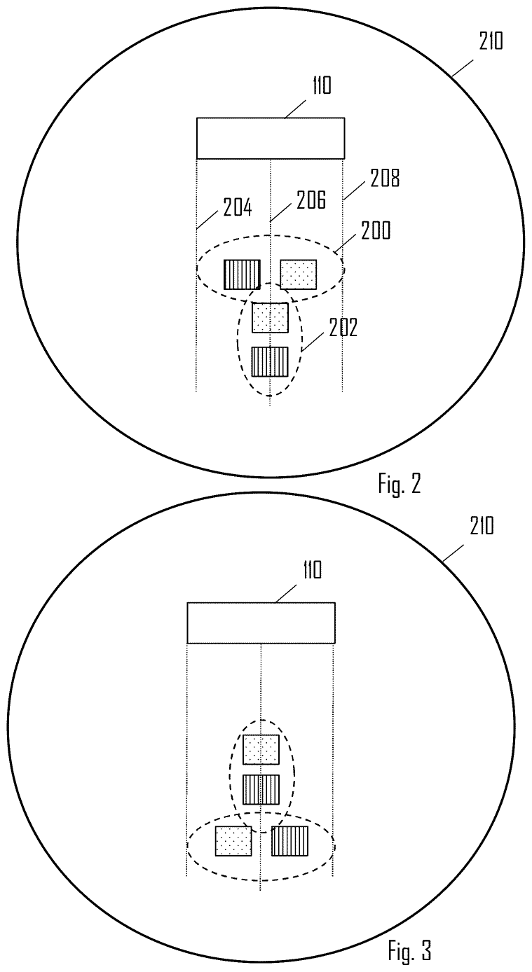

FIG. 2 illustrates the blood oximetry measurement head in greater detail. In this case, the wearable sensor device 210 is different from that of FIGS. 1A and 1B, just to illustrate that the blood oximetry measurement head according to the embodiments described herein is applicable to various wearable sensor devices. The light emitters 112 to 118 are in the same arrangement and orientation with respect to the photo detector 110 as in FIG. 1B. FIG. 2 indicates the first pair of light emitters 200 and the second pair of light emitters 202 by respective dashed circles.

As illustrated in FIGS. 1B and 2, the components 110 to 118 of the blood oximetry measurement head are arranged on the same plane, e.g. on the same substrate. The first pair of light emitters 200 is arranged in a perpendicular orientation with respect to the second pair of light emitters 202. Furthermore, the two pairs of light emitters 200, 202 form a T-shaped arrangement. Equivalently, one can see them as Y-shape because of the small number of light emitters. The T-shape is formed by the geometry of the light emitters. In many photoplethysmogram (PPG) sensors, rectangular LEDs are employed. Accordingly, the T shape may be realized by arranging the first pair of rectangular light emitters 200 side-by-side and arranging the second pair of rectangular light emitters 202 side-by-side and, then, arranging one of the pairs 200 and 202 to form one branch of the T and the other one of the pairs 200 and 202 to form the other two branches of the T, as illustrated in FIG. 2. The T-junction is thus at the intersection of an imaginary line drawn through the centers of the first pair of light emitters and an imaginary line drawn through the centers of the second pair of light emitters.

In another embodiment, the light emitters 112 to 118 are arranged on a first plane and the photo detector 110 is arranged on a second plane that protrudes from the first plane towards the skin tissue. In other words, the photo detector 110 has a closer skin contact than the light emitters 112 to 118.

The arrangement of FIG. 2 provides the advantage described above, namely that the emitters illuminate substantially the same tissue. This can be seen when looking at FIG. 2: the combined light from the emitters of the first wavelength (the dotted pattern) travels substantially along the same lateral direction to the photo detector as the combined light from the emitters of the second wavelength (the vertical lining). Because of the different wavelengths, penetration of the light into the tissue is different, thus enabling the blood oximetry measurements. In the embodiment of FIG. 2, the first and the second light emitters may switch places to have the same effect.

In an embodiment, the distance from the centre of the photo detector 110 to the midpoint between the light emitters 112, 116 of the first pair 200 is in the order of six-to-seven millimetres. Meanwhile, the distance from the centre of the photo detector to the midpoint between the light emitters 114, 118 is in the order of eight-to-nine millimetres. This may be measured along the line 206 of FIG. 2. The midpoint of the photo detector may be the centre of the photo diode 110.

In an embodiment the first wavelength is at a red-light spectrum range (about 630 to 700 nanometers, nm) and the second wavelength is at an infrared or near-infrared light spectrum range (about 700 to 2000 nm), and wherein a distance between the first light emitter and the third light emitter is smaller than a distance between the second light emitter and the fourth light emitter. Accordingly, the red-light emitters are closer to one another than the infrared light emitters.

In an embodiment, the light emitters of one of the two pairs are arranged on an imaginary first line that leads to the photo detector. This is illustrated in FIG. 2 by a dotted line 206 that travels through the second pair of light emitters and to the photo detector 110. The other pair of light emitters is then arranged on an imaginary second line that is perpendicular (aligned by 90 degrees) or substantially perpendicular (aligned by 70 to 110 degrees or by 80 to 100 degrees, for example) to the first line to form the T-shape. The first line travels between the light emitters of the other pair. The first line may travel through centers of said one of the two pairs. An embodiment can be envisaged where the other pair of light emitters is arranged on the second line that is inclined so that it is not exactly perpendicular to the first line. In this case, the T-shape is maintained by in an inclined manner.

In the embodiment of FIG. 2, the first pair of light emitters 200 is arranged between the second pair of emitters 202 and the photo detector 110. Both pairs of light emitters 200, 202 may be arranged to face the same side of the photo detector. This is illustrated in FIG. 2 such that the pairs of light emitters are disposed between the two imaginary lines 204, 208 drawn from the corners of the particular side of the photo detector towards the general direction of the pairs of light emitters 200, 202. Typically, rectangular photo detectors are used thanks to its properties of reducing ambient light noise. Accordingly, the lines 204, 208 may be perpendicular to the side that faces the pairs of light emitters 204, 208.

FIG. 3 illustrates an embodiment where the light emitters 212 to 218 form the same T-shaped arrangement as in FIGS. 1B and 2 but the T-shaped arrangement is rotated 180 degrees. Accordingly, the first pair of light emitters 200 that was closer to the photo detector than the second pair of light emitters in the embodiment of FIG. 2 is now further away from the photo detector than the second pair of light emitters. In other words, the second pair of light emitters is arranged between the first pair of emitters and the photo detector.

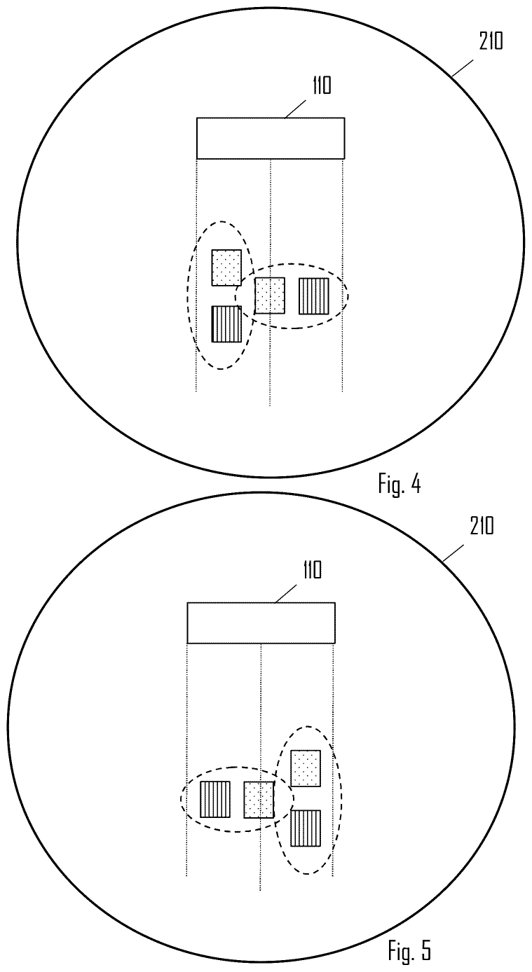

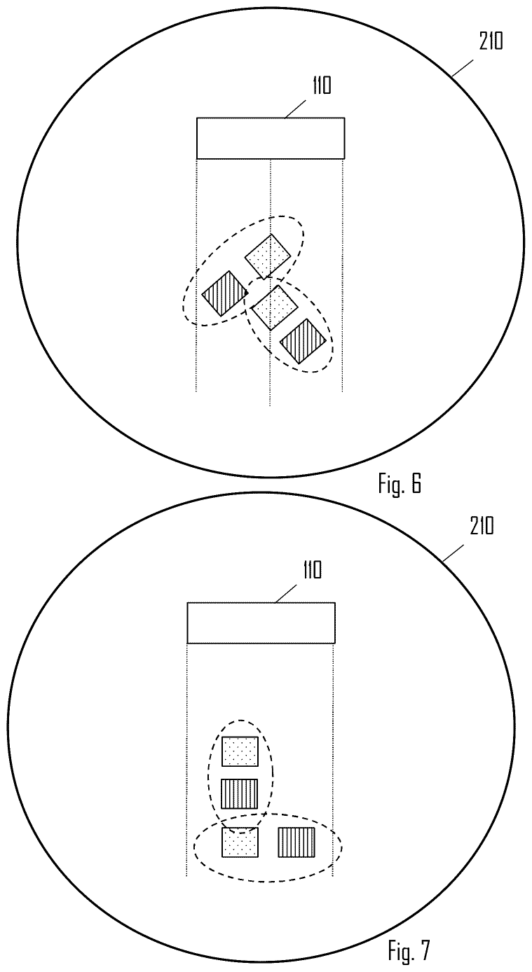

In the embodiments of FIGS. 1B, 2, and 3, the first and second pairs of light emitters are one behind the other when viewed from the photo detector 110. Furthermore, one is rotated 90 degrees with respect to the other to realize the T shape. FIGS. 4 and 5 illustrate embodiments where the first pair of light emitters 200 and the second pair of light emitters 202 are next to one another, when viewed from the photo detector 110. In the embodiment of FIG. 4, the light emitters have the same T-shaped arrangement as in FIGS. 1B, 2, and 3 but it has been rotated 90 degrees clockwise with respect to the embodiment of FIG. 3. Similarly, FIG. 5 illustrates an embodiment where the light emitters have the same T-shaped arrangement as in FIGS. 1B, 2, and 3 but it has been rotated 90 degrees clockwise with respect to the embodiment of FIG. 2. FIG. 6 illustrates yet another embodiment where the light emitters have the same T-shaped arrangement as in FIGS. 1B to 5 but it has been rotated 45 degrees clockwise with respect to the embodiment of FIG. 4. Other rotations of the light emitter arrangement are equally possible.

FIG. 7 illustrates an embodiment where the light emitters 112 to 118 are arranged in an L shape. The L shape may be rotated within the plane in the same manner as illustrated above for the T-shaped arrangement, provided that the light emitters stay between the two (imaginary) dotted lines 204, 208 extending from the photo detector, thus staying within the optimal direction with respect to the photo detector. Referring to FIG. 7, the L shape may be realized again such that the first and third light emitters are closer to one another than the second and fourth light emitters.

In all embodiments, all light emitters 112 to 118 of the blood oximetry measurement head are arranged to face the same side of the photo detector 110, i.e. they are in the same general direction from the photo detector 110. It means that the light from all the emitters 112 to 118 is received from a direction that is substantially perpendicular to the particular side of the photo detector 110. FIGS. 2 to 7 illustrate this characteristic by the dotted lines extending in parallel from the corners of that particular side towards the light emitter arrangement. As can be seen, all light emitters fit between the two lines meaning that the photo detector receives the light substantially from the same direction from all the light emitters. In an embodiment, the light emitter head of the blood oximetry sensor of the apparatus consists of the above-described T-shaped light emitter arrangement.

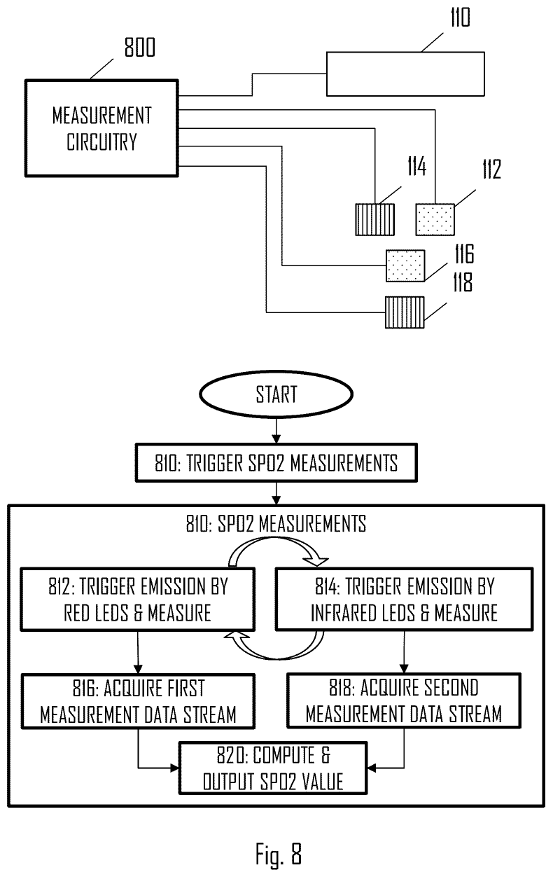

Let us then describe the above-described measurement circuitry and its operation in the conduction of the blood oximetry measurements. FIG. 8 illustrates the measurement circuitry 800 and its connections to the photo detector 110 and the first and second sets of light emitters 200, 202. FIG. 8 also illustrates a flow diagram of the operation of the measurement circuitry 800 during the blood oximetry (SpO2) measurements. The measurement circuitry may comprise at least one processor or a processing circuitry and at least one memory circuit accessible to the at least one processor or processing circuitry.

Referring to FIG. 8, the measurement circuitry 800 has a connection to each light emitter 112 to 118 in order to control emission of the light emitters of the blood oximetry measurement head. Similarly, the measurement circuitry has a connection to the photo detector 110 to carry out collection of the measurement signals representing the light received by the photo detector 110 from the light emitters emitting at a particular time, as controlled by the measurement circuitry 800.

Referring to the flow diagram of FIG. 8, the measurement circuitry 800 triggers the blood oximetry measurements in block 810. The triggering may be responsive to a user input received via a user interface, an input received from an application processor of the wearable training computer, or powering up the apparatus, for example. In block 810, the blood oximetry measurements are conducted. The blood oximetry measurements include sequential (alternating) illumination of the light emitters of the first wavelength and the light emitters of the second wavelength and respective measurements of the light with the first and second wavelengths received by the photo detector 110. As described above, the light emitters may be LEDs. In block 812, the measurement circuitry triggers the light emitters 112, 116 to emit light at the first wavelength (e.g. red light). The light emitters 112, 116 may be configured to emit light simultaneously or in an alternating order, depending on the implementation. For example, the light emitters may be provided in a parallel electric connection and, thus, they operate by using the same control signal and, thus, emit light simultaneously. In another embodiment, an analogue front-end of the measurement circuitry 800 has a separate electric connection to each light emitter 112, 116. As a consequence, the light emitters 112, 116 may be configured to emit light simultaneously or in an alternating manner, or one may even be disabled from the measurements. Accordingly, greater diversity in measurement configurations can be achieved. While the light emitters emit light, the photo detector 110 is configured to measure the light and a respective measurement signal or measurement signals is collected from the photo detector 110 and processed and stored in the memory of the measurement circuitry.

While block 812 is being executed, the light emitters 114, 118 of the second wavelength may be disabled. Thereafter, the measurement circuitry disables the light emitters 112, 116 and activates the light emitters 114, 118 to emit light at the second wavelength (block 814), e.g. infrared or near-infrared light. As with the light emitters 112, 116, the light emitters 114, 118 may be configured to emit light simultaneously or in an alternating order, depending on the implementation. For example, the light emitters may be provided in a parallel electric connection and, thus, they operate by using the same control signal and, thus, emit light simultaneously. In another embodiment, the analogue front-end of the measurement circuitry 800 has a separate electric connection to each light emitter 114, 118. As a consequence, the light emitters 112, 116 may be configured to emit light simultaneously or in an alternating manner, or one may even be disabled from the measurements. Accordingly, greater diversity in measurement configurations can be achieved. Similarly, the photo detector is configured to measure the light and a respective measurement signal is collected from the photo detector 110 and processed and stored in the memory of the measurement circuitry. The measuring and storing may be carried out according to the state-of-the-art. While block 814 is being executed, the light emitters 114, 118 of the first wavelength may be disabled (block 812 disabled). In this manner, blocks 812 and 814 may be alternately enabled and disabled to acquire (block 816) a first measurement data stream as an output of block 816 and acquire (block 818) a second measurement data stream as an output of block 814. On the basis of the first and second measurement data stream, the blood oximetry parameter representing the measured blood oxygenation may be computed in block 820. The computation may be carried out according to the state-of-the-art.

In an embodiment, all light emitters 112 to 118 may be used when computing a single value of the blood oximetry parameter. The blood oximetry may be computed repeatedly in order to get a dynamic reading.

In the embodiment where the light emitters 112, 116 are configured to emit light simultaneously, the respective measurement channels are combined readily at the photo detector into the first measurement data stream. In the embodiment where the light emitters 112, 116 are configured to emit light in an alternating manner, the photo detector 110 logically collects to separate measurement signals or measurement signal streams at respective time instants, and the analogue front-end of the measurement circuitry 800 may then combine the separate measurement signals (or signal streams) in block 816 into the first measurement data stream. The same applies to blocks 814 and 818.

In an embodiment of the process of FIG. 8, the measurement circuitry 800 is further configured to control the first light emitter 112 and the third light emitter 116 to emit light simultaneously for measuring motion of the apparatus, to control the photo detector 110 to measure the light and to generate a third measurement signal, and to compute a motion compensation parameter on the basis of the third measurement signal. Because the motion is measured from the skin surface, no deeper penetration of the light into the tissue is required, and the measurement circuitry is configured to configure a smaller light intensity for said measuring the motion than for measuring the blood oxygenation.

In summary, there is provided a wearable sensor device comprising the above-described apparatus having the T-shaped or L-shaped blood oximetry light emitter head and further comprising a strap, a band, or a garment to attach the photo detector and the first and second pairs of light emitters to a skin contact for measuring the blood oxygenation.

As used in this application, the term ‘circuitry’ refers to all of the following: (a) hardware-only circuit implementations, such as implementations in only analogue and/or digital circuitry, and (b) combinations of circuits and software (and/or firmware), such as (as applicable): (i) a combination of processor(s) or (ii) portions of processor(s)/software including digital signal processor(s), software, and memory(ies) that work together to cause an apparatus to perform various functions, and (c) circuits, such as a microprocessor(s) or a portion of a microprocessor(s), that require software or firmware for operation, even if the software or firmware is not physically present. This definition of ‘circuitry’ applies to all uses of this term in this application. As a further example, as used in this application, the term ‘circuitry’ would also cover an implementation of merely a processor (or multiple processors) or a portion of a processor and its (or their) accompanying software and/or firmware.

The techniques and methods described herein may be implemented by various means. For example, these techniques may be implemented in hardware (one or more devices), firmware (one or more devices), software (one or more modules), or combinations thereof. For a hardware implementation, the apparatus(es) of embodiments may be implemented within one or more application-specific integrated circuits (ASICs), digital signal processors (DSPs), digital signal processing devices (DSPDs), programmable logic devices (PLDs), field programmable gate arrays (FPGAs), graphics processing units (GPUs), processors, controllers, micro-controllers, microprocessors, other electronic units designed to perform the functions described herein, or a combination thereof. For firmware or software, the implementation can be carried out through modules of at least one chipset (e.g. procedures, functions, and so on) that perform the functions described herein. The software codes may be stored in a memory unit and executed by processors. The memory unit may be implemented within the processor or externally to the processor. In the latter case, it can be communicatively coupled to the processor via various means, as is known in the art. Additionally, the components of the systems described herein may be rearranged and/or complemented by additional components in order to facilitate the achievements of the various aspects, etc., described with regard thereto, and they are not limited to the precise configurations set forth in the given figures, as will be appreciated by one skilled in the art.

It will be obvious to a person skilled in the art that, as the technology advances, the inventive concept can be implemented in various ways. The invention and its embodiments are not limited to the examples described above but may vary within the scope of the claims.

What is claimed is:

1. An apparatus, for optically measuring blood oxygenation from a skin tissue, comprising:
a photo detector;
a first pair of light emitters comprising a first light emitter configured to emit light at a first wavelength and a second light emitter configured to emit light at a second wavelength different from the first wavelength;
a second pair of light emitters comprising a third light emitter configured to emit light at the first wavelength and a fourth light emitter configured to emit light at the second wavelength, wherein the second pair of light emitters is disposed in a substantially perpendicular orientation with respect to the first pair of light emitters to form a T shape or an L shape, and wherein the first pair of emitters and the second pair of emitters are to the same direction from the photo detector; and
a measurement circuitry configured to:
control the first light emitter and the third light emitter to emit light on the first wavelength, and control the photo detector to measure the light on the first wavelength and to generate a first measurement signal,
control the second light emitter and the fourth light emitter to emit light on the second wavelength, and control the photo detector to measure the light on the second wavelength and to generate a second measurement signal, and
compute a blood oxygenation parameter on the basis of the first measurement signal and the second measurement signal and to output the blood oxygenation parameter through an interface.

2. The apparatus of claim 1, wherein the first pair of light emitters is arranged in a perpendicular orientation with respect to the second pair of light emitters.

3. The apparatus of claim 1, wherein the first wavelength is at a red light spectrum range and the second wavelength is at an infrared or near-infrared light spectrum range, and wherein a distance between the first light emitter and the third light emitter is smaller than a distance between the second light emitter and the fourth light emitter.

4. The apparatus of claim 1, wherein the photo detector has a rectangular shape, and wherein the first pair of light emitters and the second pair of light emitters face one of the longer sides of the rectangular photo detector.

5. The apparatus of claim 1, wherein the first light emitter and the second emitter are arranged on an imaginary first line that leads to the photo detector, and wherein the third light emitter and the fourth light emitter are arranged on an imaginary second line that is perpendicular to the first line.

6. The apparatus of claim 5, wherein the second pair of light emitters is arranged between the first pair of emitters and the photo detector.

7. The apparatus of claim 5, wherein the first pair of light emitters is arranged between the second pair of emitters and the photo detector.

8. The apparatus of claim 5, wherein the first pair of light emitters and the second pair of light emitters are next to one another, when viewed from the photo detector.

9. The apparatus of claim 1, wherein the measurement circuitry is further configured to control the first light emitter and the third light emitter to emit light simultaneously for measuring motion of the apparatus, to control the photo detector to measure the light and to generate a third measurement signal, and to compute a motion compensation parameter on the basis of the third measurement signal, wherein the measurement circuitry is configured to configure a smaller light intensity for said measuring the motion than for measuring the blood oxygenation.

10. The apparatus of claim 1, wherein the photo detector and the light emitters are arranged on the same plane.

11. The apparatus of claim 1, wherein the light emitters are arranged on a first plane and the photo detector is arranged on a second plane that protrudes from the first plane towards the skin tissue.

12. A wearable sensor device comprising:
an comprising:
a photo detector;
a first pair of light emitters comprising a first light emitter configured to emit light at a first wavelength and a second light emitter configured to emit light at a second wavelength different from the first wavelength;
a second pair of light emitters comprising a third light emitter configured to emit light at the first wavelength and a fourth light emitter configured to emit light at the second wavelength, wherein the second pair of light emitters is disposed in a substantially perpendicular orientation with respect to the first pair of light emitters to form a T shape or an L shape, and wherein the first pair of emitters and the second pair of emitters are to the same direction from the photo detector; and
a measurement circuitry configured to
control the first light emitter and the third light emitter to emit light on the first wavelength, and control the photo detector to measure the light on the first wavelength and to generate a first measurement signal,
control the second light emitter and the fourth light emitter to emit light on the second wavelength, and control the photo detector to measure the light on the second wavelength and to generate a second measurement signal, and
compute a blood oxygenation parameter on the basis of the first measurement signal and the second measurement signal and to output the blood oxygenation parameter through an interface; and
a strap, a band, or a garment to attach the photo detector and the first and second pairs of light emitters to a skin contact for measuring the blood oxygenation.
